(19) United States
(12) Patent Application Publication
(10) Pub. No.: US 2013/0218296 A1
(43) Pub. Date: Aug. 22, 2013

(54) ADJUSTABLE PROSTHETIC SOCKET AND SUSPENSION SYSTEM

(71) Applicants: Wayne KONIUK, Belmont, CA (US); Garrett Hurley, San Francisco, CA (US)

(72) Inventors: Wayne KONIUK, Belmont, CA (US); Garrett Hurley, San Francisco, CA (US)

(21) Appl. No.: 13/757,671

(22) Filed: Feb. 1, 2013

Related U.S. Application Data
(60) Provisional application No. 61/593,774, filed on Feb. 1, 2012.

(51) Int. Cl. A61F 2/80

(52) U.S. Cl. CPC A61F 2/80
USPC 623/34

(57) ABSTRACT

The present disclosure is directed towards a socket and suspension system that offers the user increased ease of donning the prosthesis, volume adjustability, and improved circulation. The prosthetic includes a socket and liner. The socket and liner can be custom made for the amputee or prefabricated. The liner acts as an interface between the custom socket and the amputee's residual limb. Moving fluid in or out of the area between the liner and the socket causes the liner to push away from the socket wall or pull into the socket wall thereby changing the volume inside the socket. Moving the liner closer to the socket wall enlarges the volume inside the socket making it easier for the patient to put on the prosthetic. The socket and suspension system can include a pump which can manually or automatically adjust the volume inside the socket and suspension of the prosthesis.

### CROSS REFERENCE TO RELATED APPLICATIONS

This application claims the benefit of U.S. Provisional Patent Application No. 61/593,774, filed Feb. 1, 2012, the entire disclosure of which is incorporated herein by reference.

### INCORPORATION BY REFERENCE

All publications and patent applications mentioned in this specification are herein incorporated by reference to the same extent as if each individual publication or patent application were specifically and individually indicated to be incorporated by reference.

### FIELD

The present disclosure relates generally to prosthetic devices.

### BACKGROUND

A bane for amputees is a prosthesis that can be worn comfortably over a long period of time. New shoes, wear and tear, or shrinking, atrophy, swelling of the residual limb either require or lead to the necessity of adjusting the prosthetic device. Moreover, too much pressure in certain areas can lead, over time, to skin ulcers. Adjustments of an amputee's prosthetic device typically require trips to the prosthetist's office, where such adjustments to achieve a better degree of comfort and a certain degree of improved relationship between the limb and the artificial device involved the use of wrenches and fillers to make adjustments. There have been a number of prior attempts at minimizing the patient's dependency on medical professionals.

U.S. Pat. No. 3,309,714 to Porten discloses an inflatable inner liner. The inner liner is attached to the inner surface of the socket. The prosthetic limb accepts the socket which in turn accepts the residual limb. The inflatable liner is in contact with the residual limb. Air is introduced under pressure to expand the liner and provide something akin to a patient adjustable shock absorber. Inflating the liner can push the patient's residual limb out of the socket, making it more difficult to fit the prosthesis. The inflatable liner provides less support, a worse fit, and a poorer quality seal with the residual limb than the devices and methods disclosed herein.

U.S. Pat. No. 5,108,456 to Fay discloses a plurality of bladders attached to the outer surface of the socket. Through a series of valves and conduits, the bladders can be selectively inflated or deflated. When inflated the bladder or bladders applies pressure to a selected portion or portions of the socket to force that portion or portions of the socket inwardly to grip the residual limb.

U.S. Pat. No. 5,724,714 to Love discloses a composite socket. This composite socket contemplates an outer socket affixed to an artificial limb component and having an inner cavity for receiving an inner socket which is adapted to receive the residual limb. A bladder is nestled between the inner and outer sockets. The bladder has a user operated pump to inflate the bladder.

The aforementioned prior art inflatable liners or bladders risk over inflation by the patient and damage to the tissue of the residual limb. Therefore, other attempts include the suggestion of improved volume adjustment to minimize this risk. One such suggestion, for example, is a self-inflating socket disclosed in U.S. Pat. No. 6,149,691 to Fray. In this prior configuration the socket has an inner part, an outer part and gelatinous material there between. The gelatinous material absorbs shock encountered by the amputee when walking. A self-inflating, volume-adjusting bladder is also positioned in the socket and has a valve mechanism that allows air to flow into and out of the bladder as the volume of the residual limb changes.

The common approach utilized by these prior art attempts is to have the interface liner and socket as separate units. Separate sealing sleeves, also known as suspension sleeves, or sealing rings on the liner or the wall of the socket to achieve suspension. The present disclosure varies from prior art attempts in that the interface liner and socket comprise one complete socket and suspension system.

Prior art devices can also be difficult to put on the residual limb. The devices disclosed herein are a significant improvement for amputees who lack the strength, dexterity, or the ability to pull into or push into a suction socket. The devices also provide a system that has improved comfort and increased protection of the skin over the prior art. These properties are especially valuable because over 50% of amputations are caused by complications resulting from diabetes and people with diabetes need to be especially careful at protecting their skin. In addition, many people with diabetes have poor hand strength.

The devices disclosed herein make it easier to don the prosthesis, have improved volume adjustability, improved limb circulation, and additional benefits over preexisting prosthetic devices.

### BRIEF SUMMARY

Prosthetic systems for suspending a prosthesis from a patient's residual limb are disclosed herein. The systems can include a socket having an open end, an inner wall, an outer wall, and a brim defining the open end. The systems can also include a liner integrated with the socket and forming a seal with the socket. The liner can at least partially define a sealed space between the liner and the inner wall of the socket. The liner is moveable between a first position in which is it adapted to engage the patient's residual limb and a second position closer to the inner wall of the socket than the first position.

The systems can also include a fluid communication channel between the sealed space and the outer wall of the socket. The systems can also include a pump configured to engage with the fluid communication channel. The pump can be a negative and positive pressure pump. The pump can include a control system. The control system can be configured to control the pump to adjust a pressure and a volume in the sealed space. The control system can include sensors such as a pressure sensor, an accelerometer, and a gyroscope.

The sealed space can contain a fluid. The liner can be sealed to the interior wall of the socket adjacent to the brim. The liner can be reflected over the brim and sealed against the outside wall of the socket. The prosthetic system can also include a distal retaining member configured to adjust a depth of the patient's residual limb within the liner and socket.

Methods for donning and suspending a prosthesis from a patient's residual limb are disclosed herein. The methods can include providing a socket comprising a liner and a sealed space containing fluid between the socket and liner, removing a portion of the fluid, inserting the patient's residual limb into the liner and socket, introducing fluid into the sealed space to move the liner against the patient's residual limb, and suspending the prosthesis attached to the socket. The methods can also include adjusting the depth of the residual limb within the liner and socket. The methods can also include pulsing the fluid into and out of the sealed space to aid in blood circulation of the patient's residual limb. Pulsing the fluid can be performed using a pump.

### BRIEF DESCRIPTION OF THE DRAWINGS

FIG. 1 is a cross-sectional view of a residual limb suspended within a socket in accordance with an embodiment.

FIGS. 2 and 3 show cross-sectional view of a socket in accordance with an embodiment.

FIG. 4 is a cross-sectional view of a socket in accordance with an embodiment.

FIG. 5 is a cross-sectional view of a socket in accordance with an embodiment.

FIG. 6 is a side view of a socket in accordance with an embodiment.

FIG. 7 is a picture of a socket in accordance with an embodiment.

FIG. 8 is a picture of a socket attached to a prosthesis in accordance with an embodiment.

FIGS. 9A-9C illustrate schematic cross-sectional views of various configurations of sockets in accordance with embodiments.

### DETAILED DESCRIPTION

Improved methods and apparatuses for suspending a prosthesis are disclosed herein. The systems include a socket and liner offer volume and shape adjustability of the interior space in the socket. The system offers increased ease of donning, improved circulation, and automated volume control.

The socket has an inner volume that is capable of receiving the patient's residual limb. The socket can include a brim along an opening of the socket. The socket can also include a liner integrated with the socket in the inner volume of the socket. The liner and socket can define a sealed space between the liner and socket. The liner can be flexible and stretchable. The liner can be designed to contact the skin of the residual limb. The liner can be designed of a comfortable material that can form a seal with the patient's residual limb.

The socket can be custom made for the amputee. In some embodiments the socket can be prefabricated. The liner for the socket can be custom made or prefabricated. In some embodiments the liner can be separate from the custom socket and sealed to the custom socket.

The sockets disclosed herein can be used with any type of prosthesis. For example, the prosthetic devices can be used on the upper or lower extremities. In some embodiments the devices disclosed herein are used for residual limbs such as arms amputated above or below the elbow. In some embodiments the devices disclosed herein are used for residual limbs such as legs amputated above or below the knee. The systems disclosed herein can be used with prosthetic arms, hands, legs, and feet. The socket can include a mechanical means for attaching the prosthesis. Examples include a threaded screw receptacle, threaded screw, and other mechanical attachment structures that engage with the prosthesis.

In some embodiments the liner is sealed to the socket. The liner can be sealed to the socket using a silicone adhesive or other suitable sealant material. The sealed space can include vapor (e.g. air), gel, or other suitable fluid between the interface liner and the socket. Sealing the liner to the socket can prevent air from going into and out of the socket. Sealing the liner to the socket can allow for an improved seal between the patient's residual limb and the socket because the liner can contact a higher surface area of the residual limb versus sockets that use multiple inflatable bladders.

The interface liner can be made of a suitable pliant material. Examples of pliant materials include silicone, thermoplastic elastomer (TPE), urethane, or other suitable material. The liner can include a material or coating that improves the strength of the liner and reduces the likelihood of puncturing or tearing. The liner can include a tacky inner liner material that can improve the contact and seal with the patient's residual limb. The liner can be made of a hypoallergenic material or can have a hypoallergenic coating or surface. The liner can include an antibacterial coating or material.

In some embodiments the interface liner is pulled over the proximal brim of the socket and attached to the exterior of the socket. Sealing the liner to the socket, such as by sealing the interface liner to the interior or exterior of the socket, serves to seal air from getting into and out of the socket. Additionally, pulling the liner over the brim can provide more comfort and skin protection at the brim because the liner wraps around the brim of the socket and the materials used for the interface liner are compliant and soft.

In some embodiments the liner can be sealed to the interior of the socket. For example, a sealing section can be made such that the liner is sealed against the inside of the socket adjacent to the brim. The patient's residual limb can engage with the inner liner to form a seal with the socket and liner.

In some embodiments the socket and liner can include a brim guard to protect the liner and brim area from damage, including any portion of the liner adjacent to the brim of the socket. The brim guard can include additional padding.

In some embodiments a pump is used with the socket and liner. For example, a positive and negative pressure air pump can be provided for manually or automatically moving the air, gel, or other suitable fluid in or out of the sealed space between the interface liner and the socket. The volume in the sealed space can be modified by adding or removing fluid from the sealed space. Adding fluid to the sealed space causes the interface liner to push away from the socket wall towards the interior of the socket. Removing fluid from the sealed space pulls the liner towards the socket wall thereby increasing the interior volume inside the socket. Pulling the liner towards the socket wall can be utilized to expand the volume before donning the prosthesis for ease of donning.

The ability to enlarge the socket volume can make donning the prosthesis much easier for many patients, especially patients with poor dexterity and strength. The ease of donning the prosthesis is a big improvement over conventional prosthesis sockets, that don't have an expandable interior volume because the tight fit makes it harder for the patient to fit the residual limb into the prosthesis. Prior art sockets can require modifications or preparation of a new custom socket for volume changes to the patient's residual limb. The ability to change the volume in the socket can also greatly reduce maintenance on the socket and trips to the medical office.

Once the residual limb is in the socket, the interface liner can then be allowed to wrap around the residual limb for suspension and total contact. The flexible liner can contact a greater surface area of the patient's residual limb. The liner and sockets disclosed herein offer an improved seal with the patient's residual limb. The improved seal between the patient's limb and the liner and socket improves the comfort for the patient and decreases the likelihood of losing the seal and having the prosthetic become loose or fall off. The devices disclosed herein can reduce the likelihood of the prosthetic device becoming loose or falling off the patient. The liner material, thickness, elasticity, size, and configuration can be selected such that the liner contacts the patient's residual limb with a desired contact pressure. The liner can apply an evenly distributed contact pressure to the residual limb. The ability to allow the liner to wrap around the residual limb is an improvement over the use of inflatable bladders. Inflating a bladder within the socket can push the patient's residual limb out of the socket. The inflatable bladder can also result in uneven pressure applied to the residual limb. The uneven pressure can make the socket less comfortable, cause a poor fit, and result in a weaker seal with the residual limb.

In some embodiments a pump and control system can be used to monitor the prosthetic device. For example, the pressure, volume, and resulting shape inside the socket can be monitored and regulated with a specialized pump having an automated microprocessor control and sensors. Examples of sensors include but are not limited to one or more of the following; gyroscope, accelerometer, and/or a pressure sensor. The pump and control system can include a pulse function. The pulse function for the pump can vary the pressure inside the socket and can be utilized to improve circulation. The pulse function can include pulsing between positive and negative pressure. If the amputee's residual limb decreases or increases in volume or shape throughout the day or over time, the liner and pump system can expand or contract to accommodate these changes. In some embodiments the socket can have volume increase sections in non-critical areas while still having control of the bony anatomy in the critical biomechanical control areas. In some embodiments the socket can have volume decrease sections in non-critical areas.

In some embodiments the pump can be specially designed and fabricated for use with the socket. The pump may or may not be removable. The pump can attach to the socket. In some embodiments the pump is used to expel air out of the sealed space and passively allow air into the sealed space.

A negative and positive air pressure pump can offer the ability to vary pressure to the appropriate amount for the given patient with manual, internal, or external power options. A negative and positive pressure air pump can also provide an oscillating feature that slightly alternates between positive and negative pressure to aid in circulation for the residual limb. An oscillating pressure feature can be beneficial for the large amount of amputees who have poor circulation in that it simulates muscle action and typically offers more aid to circulation.

In some embodiments the pump can include manual or automated controls with the ability to set a desired amount of pressure in the system. In some embodiments the amount of pressure in the system can be shown by a mechanical or digital gauge. In some embodiments a microprocessor control coupled with the appropriate sensor devices disclosed herein can be used to automatically adjust volume and pressure for when the patient is walking, running, or sitting. The volume and pressure can be automatically optimized based on the type of patient activity.

The sealed space can be in fluid communication with an exterior wall of the socket via a fluid communication channel. The pump can engage with the fluid communication channel to move fluid into and out of the sealed space. In some embodiments one or more fluid communication channels can be used.

In some embodiments the sealed volume between the inner liner and socket can include multiple discrete volumes. The multiple discrete volumes can include two or more separate sealed volumes. Each of the sealed volumes can be in fluid communication with an exterior of the socket using one or more fluid communication channels.

In some embodiments a distal retainer member and sealing ring can be integrated into the inside of the interface liner. The distal retainer member can be depth adjustable. The distal retainer member, with or without depth adjustability, retains the interface liner distally by affixing the interface liner to the distal portion of the socket. This distal retaining member prevents the interface liner from inverting. A depth adjustability feature of the distal retainer member can be included to allow for the distal retainer to increase and decrease the depth of the socket as the patient's limb changes in volume while still remaining affixed to the distal portion of the socket. This depth adjustability feature can have automated or manual control. The depth adjustability feature would be beneficial in situations where the patient's residual limb has shape changes that include a change in the length of the residual limb.

In some embodiments the socket includes a suction socket valve and valve housing. The socket valve and housing can be secured to a distal portion of the interface liner and to a distal portion of the inner socket. The valve housing can also allow for the suction valve to be removed and reinserted. For example, the patient could pull into the socket with use of a prosthetic donning bag or pull bag.

In some embodiments the socket utilized for the present suspension system with volume control can be oversized to the residual limb in select areas of the socket to allow for increased ability for the system to accommodate increases in residual limb volume and to allow for increased ease of donning. For example, the socket can be oversized in non-critical areas to provide extra space in areas that are not required for biomechanical control of the residual limb. In this manner, the socket can allow for volume expansion but still offer biomechanical control. The amount to oversize the socket in select areas can be standard or based on the typical amount of volume fluctuation that the particular amputee experiences based on the patient type, type of amputation, and residual limb features.

In some embodiments, the interface liner can be undersized to the residual limb so that the system accommodates for volume loss. Therefore, if the patient's residual limb gets smaller, the interface liner is capable of reducing in size and shape with the amputee's residual limb. Having the interface liner undersized to the residual limb also creates the ability for increased suspension. The flexibility of the liner allows for the prosthetic device to adjust for volume changes in the patient's residual limb. The volume flexibility can save the patient from additional trips to a medical office for resizing of the socket or requiring a new socket to be custom made to account for the volume changes.

Any of the embodiments described herein can also include an emergency release valve to allow for manual removal of the prosthesis from the amputee's residual limb without requiring operation of the pump.

In some embodiments a lamination spider or wicking channels can be incorporated into the socket or the liner to facilitate the continued ability to evacuate air from the suspension system without the interface liner sealing against the inner wall of the socket and blocking air flow.

Unless otherwise indicated, all numbers used in the specification and claims are to be understood as being modified in all instances by the term “about.” Accordingly, unless indicated to the contrary, the numerical parameters set forth in the specification and attached claims are approximations that may vary depending upon the desired properties.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Terms such as “interface liner,” “liner,” “reflected liner,” or “reflective liner,” for the purposes of describing elements of the invention, are intended to be construed as interchangeable. Terms such as “pull bag” or “donning bag” are intended to be similarly construed.

The following detailed description should be read with reference to the drawings in which similar elements in different drawings are numbered the same. The drawings, which are not necessarily to scale, depict illustrative embodiments and are not intended to limit the scope of the claims.

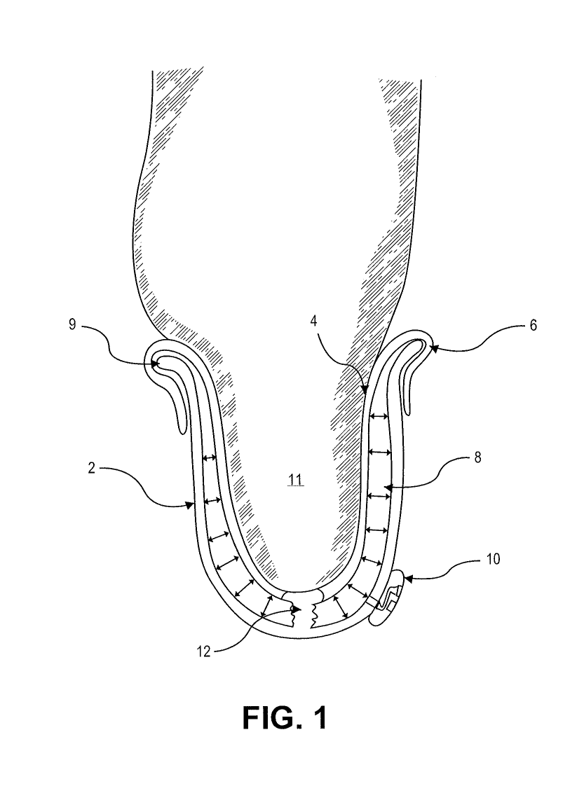

FIG. 1 represents an embodiment of a prosthetic suspension system. In this embodiment residual limb 11 is suspended within socket 2. A reflected liner 6 has an inner portion 4. Inner portion 4 is within socket 2 in contact with residual limb 11. Liner 6 reflects over the socket brim 9 and partially around the socket 2. The socket 2 and liner 6 define a space 8 between the inner portion 4 of the liner 6 and the inner wall of the socket 2. A positive and negative pressure pump 10 communicates with the space 8 existing between socket 2 and reflective liner 6.

Interface liner 6 can be fabricated from silicone, thermoplastic elastomer, urethane or other materials suitable to protect the liner from being punctured or torn. Additionally, a brim guard can be provided to further avoid punctures. Liner 6 can also be provided with a tacky inner liner material that assists in maintaining suspension of the socket. This inner liner material may also have hypoallergenic and antibacterial coatings or surface medications.

Space 8 between interface liner 6 and socket 2 can contain air, gel or other suitable fluid. Use of a fluid or gel would have the additional advantage of giving better suspension and control of the prosthesis. Use of a fluid or gel, however, would require a different pump and a reservoir for any volume of fluid or gel that is taken out of space 8.

The negative and positive pressure pump 10 provides volume and shape adjustability. The system, therefore, is able to suspend a prosthesis (not shown) from the tension of reflective liner 6 and the friction of reflected liner 6 against the skin of residual limb 11. The positive and negative pressure pump 10 can control the reflective liner inner portion 4 such that it stays snug against residual limb 11, applying sufficient pressure against the residual limb 11 to hold the socket and attached prosthesis firmly in place without injury to the residual limb from too much pressure. FIG. 1 shows an adjustable distal retaining member 12 that can be used to adjust the depth of residual limb 11 within socket 2.

Pump 10 can have additional features such as an accelerometer, pressure sensors, and/or gyroscope or other appropriate sensor device incorporated therein which, along with a microprocessor, could be used to automatically adjust volume and pressure for when the patient is walking, running, or seated. In some embodiments, the pump 10 can include a pulse feature which can aid in circulation. In addition, sensors inside pump 10 could be utilized to determine the pressure of the air, gel, or fluid within the system's volume adjustable section, as well as gauge the current volume inside the socket. Power for pump 10 could be provided in any number of appropriate ways including but not limited to, chargeable batteries, removable batteries, solar charging capabilities, or any other suitable power options.

In some embodiments the pump 10 includes a pump that expels air out of the system as necessary and passively allows air into the suspension system in a controlled manner. In some embodiments a manual pump is used. A manual pump has the advantage of not requiring external power. This manual pump could, for example, operate on a small removable or attached hand pump and alternating or reversible valve option to change the direction of fluid flow.

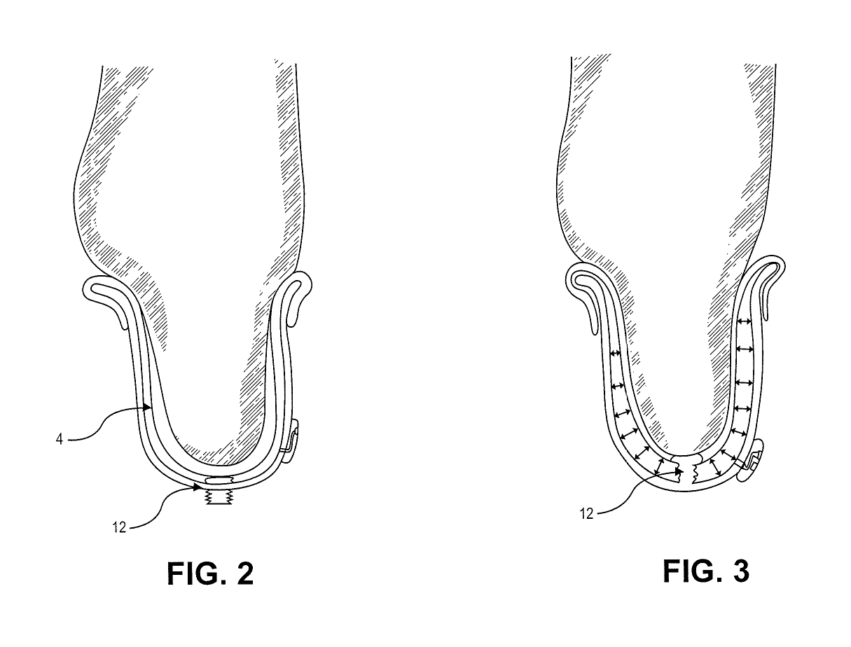

FIGS. 2 and 3 indicate cross sectional side views of an alternate embodiment. In this embodiment, donning of the prosthesis is accomplished by simply pushing into the socket along with the use of a suitable lubricant such as a prosthetic lotion. Inner portion 4, of the reflected liner, is pulled up against the socket wall during donning. As with the embodiment of FIG. 1, similarly positive and negative pressure pump 10 provides adjustability of volume in the space created between inner reflective liner portion 4 and socket 2, by control of pump 10. The distal retaining member 12 is shown in the lowest position in FIG. 2 to allow maximum depth within the suspension system. FIG. 3 depicts distal retaining member 12 having been adjusted to meet the end of residual limb 11.

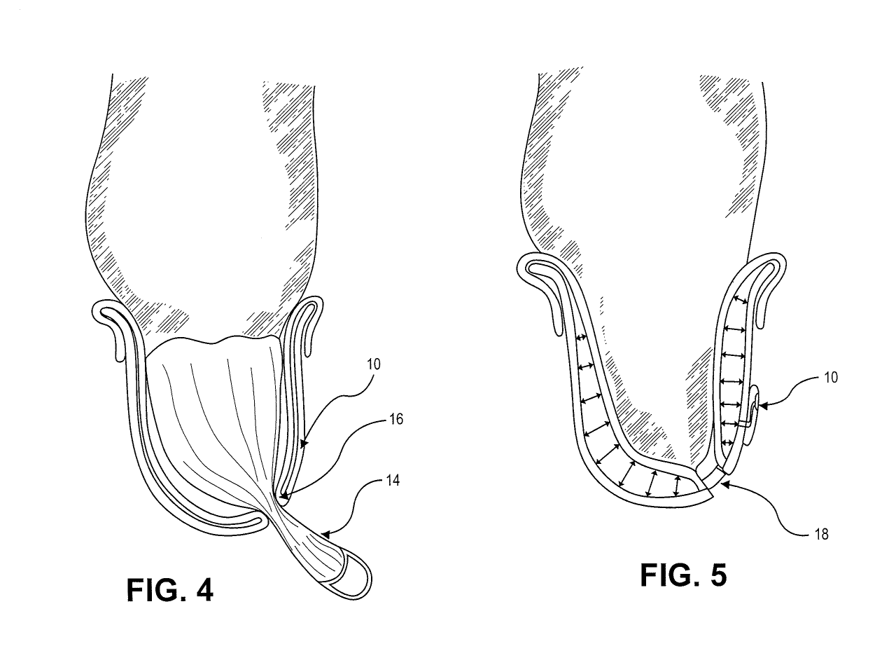

FIG. 4 represents a cross sectional side view of another embodiment. In this embodiment an alternate method of donning is presented. Inner portion 4 of reflective liner 6 is pulled against the wall of socket 2. A prosthetic pull bag 14 is used to assist in donning of the socket and prosthetic device. Prosthetic pull bag 14 is drawn through an orifice 16 in socket 2 assisting the residual limb into socket 2. The liner can be sealed to the wall of the socket 2 such that one or more sealed spaces are used. The one or more sealed spaces can be separate from each other and not in direct fluid communication.

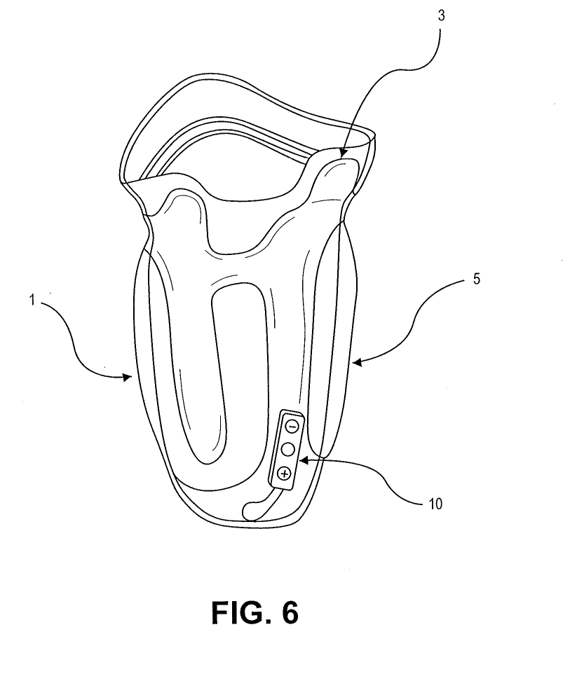

FIG. 5 illustrates another embodiment of the socket. In this embodiment a suction valve 18 is inserted into orifice 16 after pulling the residual limb into socket 2 via prosthetic pull bag 14. Suction valve 18 seals off air from the system after pulling the residual limb into socket 2. A positive and negative pump 10 could then be used to allow air back into space 8 such that the inner portion 4 of the reflected liner could retract securely onto the residual limb.

FIG. 6 is a perspective illustration of socket and suspension system. Like reference numerals are as previously stated with reference to FIGS. 1 through 5. To don the prosthesis, pump 10 is used to exhaust or move fluid from space 8 drawing inner portion 4 of the reflected liner against the inner walls of socket 2 maximizing the volume inside socket 2. The patient then easily inserts their residual limb 11 into socket 2. Using a prosthetic lotion as a lubricant will lower friction as the residual limb slides into the socket. The patient then activates pump 10 to introduce fluid into the system until the liner pulls snug against their residual limb and suspends the prosthesis.

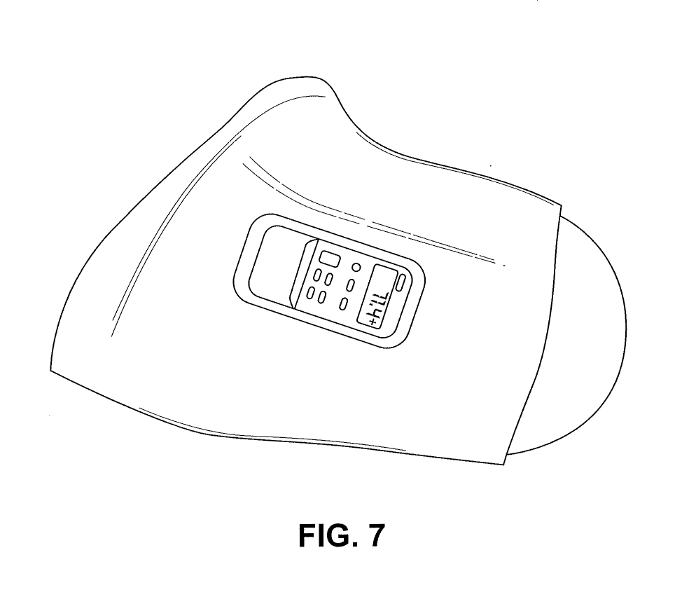

FIG. 7 is a picture of a socket and pump control system in accordance with an embodiment. The pump control system attaches to the socket.

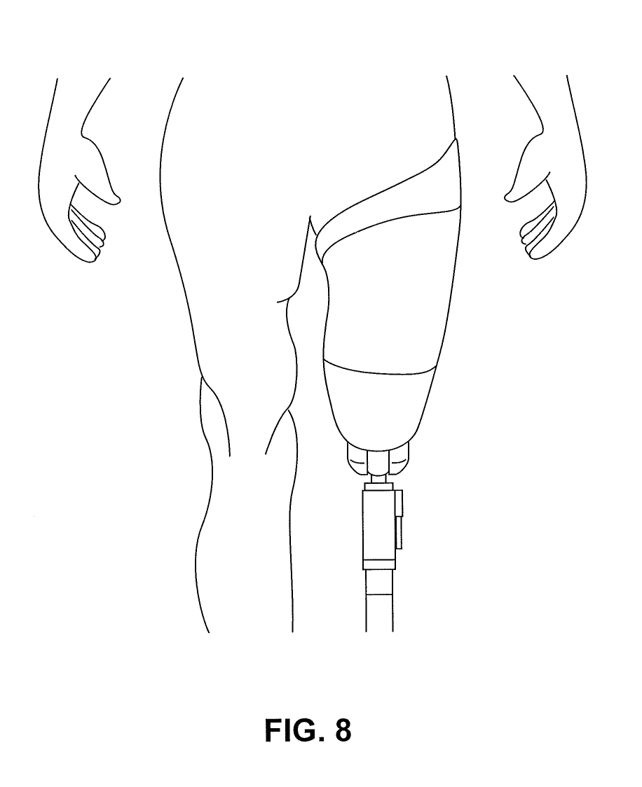

FIG. 8 is a picture of a socket attached to a prosthesis in accordance with an embodiment. The socket is attached to a prosthetic leg.

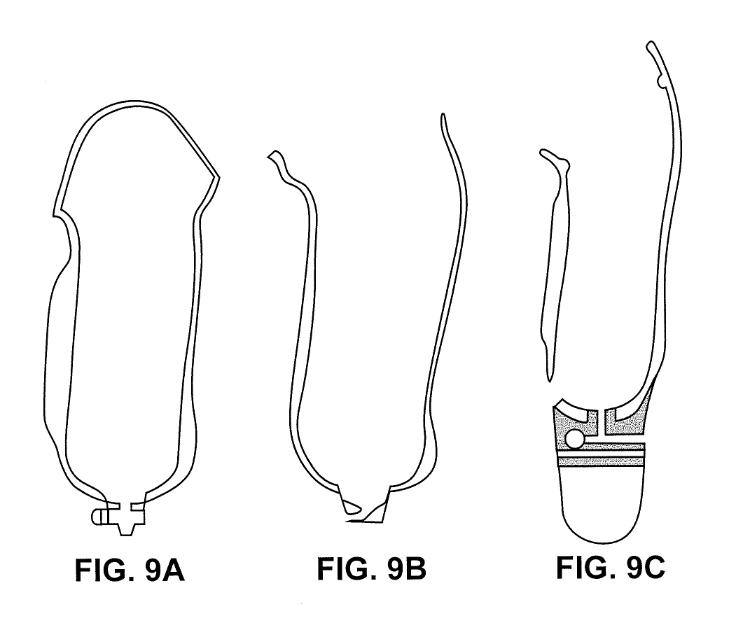

FIGS. 9A-9C are illustrations of sockets that can be used to receive different types of residual limbs.

Certain embodiments are described herein, including the best mode known to the inventors. Of course, variations on these described embodiments will become apparent to those of ordinary skill in the art upon reading the description. For example, while the description has primarily focused on the field of prosthetics, alternate embodiments could be utilized in fields such as wheelchair cushions, bike seats, orthotics, exoskeleton systems, burn masks, medical devices, and other appropriate applications. For example, it is important that burn masks maintain even pressure and total contact. The devices and methods disclosed herein could be modified to be used as burn masks with and the ability to adjust volume and fit for burn masks. Volume fluctuations can be an issue for patients using braces. The methods and devices disclosed herein are applicable to patients using braces and can use the volume adjustable suspension system to account for volume fluctuations.

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen in order to best explain the principles of the technology and its practical application to thereby enable others skilled in the art to best utilize the technology in various embodiments and with various modifications as are suited to the particular use contemplated. The present invention descriptions are intended to cover such alternatives, modifications, and equivalents as may be included within the spirit and scope of the invention as defined by the appended claims and otherwise appreciated by one of ordinary skill in the art.

What is claimed is:

1. A system for suspending a prosthesis from a patient's residual limb comprising:
a socket having an open end, an inner wall, an outer wall, and a brim defining the open end;
a liner integrated with the socket and forming a seal with the socket, the liner at least partially defining a sealed space between the liner and the inner wall of the socket, wherein the liner is moveable between a first position in which is it adapted to engage the patient's residual limb and a second position closer to the inner wall of the socket than the first position; and
a fluid communication channel between the sealed space and the outer wall of the socket.

2. The system of claim 1, further comprising a pump configured to engage with the fluid communication channel and to pump fluid into the sealed space.

3. The system of claim 2, wherein the pump is a negative and positive pressure pump.

4. The system of claim 3, wherein the negative and positive pressure pump includes a pulse function to aid blood circulation in the patient's residual limb.

5. The system of claim 2, further comprising a pump control system configured to control the pump to adjust a pressure and a volume in the sealed space.

6. The system of claim 5, wherein the pump control system comprises one or more of a pressure sensor, an accelerometer, and a gyroscope.

7. The system of claim 2, wherein the pump is a manual pump with a reversible valve.

8. The system of claim 1, wherein a fluid is contained within the sealed space.

9. The system of claim 8, wherein the fluid is air.

10. The system of claim 1, wherein the sealed space contains a gel.

11. The system of claim 1, wherein the liner is sealed to the interior wall of the socket adjacent to the brim.

12. The system of claim 1, wherein the liner is reflected over the brim and sealed against the outside wall of the socket.

13. The system of claim 1, further comprising a suction valve engaged with the fluid communication channel.

14. The system of claim 1, further comprising a distal retaining member configured to adjust a depth of the patient's residual limb within the liner and socket.

15. The system of claim 1, further comprising a limb prosthesis coupled to the socket.

16. The system of claim 1, wherein the inner section of the liner includes a sealing surface configured to form a seal with the patient's residual limb when the liner is in the first position.

17. The system of claim 1, wherein the socket includes an oversized portion configured to accommodate increases in the size of the patient's residual limb.

18. A method for donning and suspending a prosthesis from a patient's residual limb, the method comprising:
providing a socket comprising a liner and a sealed space containing fluid between the socket and liner;
removing a portion of the fluid;
inserting the patient's residual limb into the liner and socket;
introducing fluid into the sealed space to move the liner against the patient's residual limb; and
suspending the prosthesis attached to the socket.

19. The method of claim 18, further comprising adjusting the depth of the residual limb within the liner and socket.

20. The method of claim 19, further comprising pulling a vacuum on the socket to draw the patient's residual limb deeper into socket.

21. The method of claim 18, further comprising pulsing the fluid into and out of the sealed space to aid in blood circulation of the patient's residual limb.

22. The method of claim 21, wherein pulsing is performed using a pump.

23. The method of claim 18, wherein removing a portion of the fluid is done using a pump.
